(19) United States
(12) Patent Application Publication
(10) Pub. No.: US 2006/0030911 A1
(43) Pub. Date: Feb. 9, 2006

(54) Stabilization of aortic iliac neck diameter by use of radio frequency

(75) Inventor: Michel Letort, Prevessins (FR)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/921,681

(22) Filed: Aug. 18, 2004

Related U.S. Application Data
(60) Provisional application No. 60/598,340, filed on Aug. 3, 2004.

(51) Int. Cl. A61F 2/00; A61F 7/12

(52) U.S. Cl. 607/101; 607/113

(57) ABSTRACT

A radio frequency probe is positioned in a body lumen, and the body lumen is heated, using the radio frequency probe, to stabilize the body lumen for insertion of a stent graft or stent. Specifically, the heating shrinks a diameter the body lumen. In one example, blood is allowed to flow through the body lumen during the heating, and in another example, blood flow is blocked through the body lumen during the heating. Another method inserts a metallic mesh in an aneurysmal sac of an aortic aneurysm and expanding the metallic mesh to contact a wall of the aneurysmal sac. The metallic mesh is used to electro-coagulate blood from a Type II endoleak.

### RELATED APPLICATIONS

This application claims the benefit of U.S. Provisional Patent Application No. 60/598,340 filed Aug. 3, 2004.

### BACKGROUND OF THE INVENTION

1. Field of the Invention

The present invention relates generally to devices and methods to treat aortic aneurysms, and more particularly to endografts used to treat aortic aneurysms.

2. Description of the Related Art

Endografts, sometimes called stent grafts, are widely used in the treatment of aortic aneurysms. Typically, a stent graft positioned in an abdominal aortic aneurysm has a proximal end that is positioned one to two centimeters below the lowest renal artery. A bifurcated stent graft has two legs that extend into the femoral arteries.

The proximal end of the stent graft has at least one and one and a half centimeters in contact with healthy fibrous tissue of the aorta. This at least one to one a half centimeters contact area is used to form a seal between the stent graft and the aorta wall so that blood is passed through the stent graft and not into the aneurysm. Unfortunately, it has been observed that after placement of a stent graft in the aorta, the diameter of the aorta dilates about 0.5 mm per year. To maintain the seal, the stent graft must remain in contact with the wall of the aorta. One current solution to this problem is to oversize the diameter of the stent graft. Another solution is to use hooks and barbs on the stent graft to assure fixation of the stent graft to the aorta.

Unfortunately, neither of these solutions is satisfactory for all situations. For example, a stent graft with hooks and barbs is difficult to move, if mis-deployed.

Another problem that is encountered following placement of the stent graft is endoleaks. The endoleaks permit blood to refill the aneurysm that in turn presses against the weakened area of the aorta, which in turn may result in bursting of the aneurysm.

### SUMMARY OF THE INVENTION

The prior art problems associated with using a stent graft or a stent in a body lumen are reduced by positioning a radio frequency probe in the body lumen, and heating the body lumen, using the radio frequency probe, to stabilize the body lumen for insertion of the stent graft or stent. Specifically, the heating shrinks a diameter of the body lumen. For example, the body lumen is heated to a temperature greater than 60 C. In one example, blood is allowed to flow through the body lumen during the heating, and in another example, blood flow is blocked through the body lumen during the heating.

The body lumen may be any vein or artery and is for example, an aortic neck of an aortic aneurysm. The body lumen also may be an aneurysmal sac of an aortic aneurysm. Optionally, a metallic mesh is deployed in the aneurysmal sac prior to the heating.

Another method inserts a metallic mesh in an aneurysmal sac of an aortic aneurysm and expands the metallic mesh to contact a wall of the aneurysmal sac. The metallic mesh is used to electro-coagulate blood from a Type II endoleak. The method includes delivering a stent graft to the aortic aneurysm. The step of expanding the metallic mesh includes injecting a filler material into the aneurysmal sac.

### BRIEF DESCRIPTION OF THE DRAWINGS

FIG. 1 is an illustration of stabilizing a body lumen using a radio frequency heat source.

FIG. 2A is an illustration of stabilizing a neck of an aortic aneurysm using a radio frequency heat source without blocking blood flow through the aorta.

FIG. 2B is an illustration of a stabilized neck of an aortic aneurysm after using a radio frequency heat source.

FIG. 2C is an illustration of an endograft inserted in an aortic aneurysm with a stabilized neck.

FIG. 3 is an illustration of stabilizing a neck of an aortic aneurysm using a radio frequency heat source while blocking blood flow through the aorta.

FIG. 4 is an illustration of stabilizing a neck of an aortic aneurysm using a radio frequency heat source in a specified liquid environment.

FIG. 5 is an illustration of stabilizing a neck of an aortic aneurysm using a radio frequency heat source using temperature sensors.

FIG. 6 is an illustration of stabilizing an aneurysmal sac of an aortic aneurysm using a radio frequency heat source.

FIG. 7A is an illustration of a metallic mesh inserted in an aneurysmal sac of an aortic aneurysm.

FIG. 7B is a illustration of a metallic mesh inserted in an aneurysmal sac of an aortic aneurysm and then stabilizing the aneurysmal sac of the aortic aneurysm using a radio frequency heat source.

FIG. 8 is an illustration of a metallic mesh having a fabric backing inserted in an aneurysmal sac of an aortic aneurysm.

FIG. 9A is an illustration of deploying a metallic mesh in an aneurysmal sac of an aortic aneurysm so that a stent graft can be delivered.

FIG. 9B is an illustration of a deployed metallic mesh in an aneurysmal sac of an aortic aneurysm with the stent graft delivered.

FIG. 10 is a process flow diagram for the deployment of the metallic mesh and the stent graft in an aortic aneurysm.

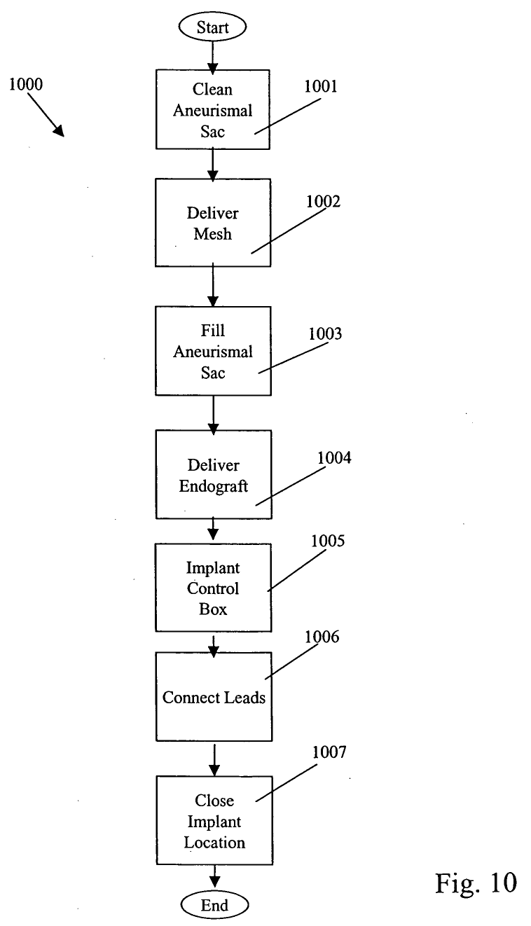

In the Figures, the first digit of a reference numeral in Figures having a single digit figure number and the first two digits of a reference numeral in the Figure having a double digit figure number are the figure number in which the corresponding element first appears.

### DETAILED DESCRIPTION

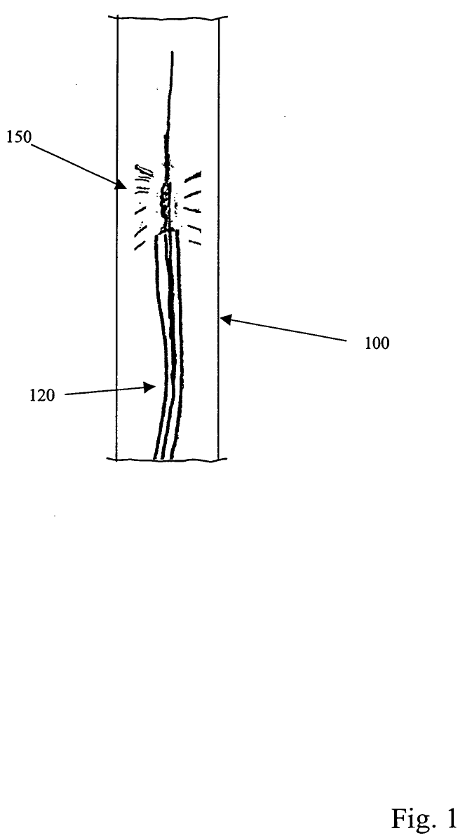

To reinforce and/or shrink a body lumen 100 (FIG. 1), a radio frequency (RF) heat source 150 is inserted in body lumen 100 using a catheter 120. RF heat source 150 is configured to transmit RF energy that heats body lumen 100 in the vicinity of heat source 150.

Body lumen 100 is heated by the transmitted RF energy for a sufficient time that collagen fibers in the wall of body lumen 100 reach a temperature in a range of 60° C. to 95° C. It is known that Type I collagen and Type III collagen are abundant in the walls of blood vessels. It has been reported that veins and arteries have 88% and 28% of their dry weight as collagen, respectively.

The heating of lumen 100 causes unwinding of the collagen triple helix and loss of collagen fiber orientation in the wall of lumen 100. In turn, Types I and III collagen contract into a shortened state that results in shrinkage of lumen 100. This shrinkage stabilizes lumen 100 so that a stent and/or stent graft placed in lumen 100 forms a more permanent contact with lumen 100 than the unstabilized lumen would. Also, with lumen 100 stabilized, problems associated with too much over-sizing of a stent graft, which can lead to overlapping of the cover material and leakage or narrowing, are minimized. Accordingly, the prior art problems are mitigated. In addition, to the shrinking that reinforces the lumen, fibroblast activity may also be stimulated, which further enhances the results of the heat treatment.

In the following description, an abdominal aortic aneurysm and use of a stent graft to treat an abdominal aortic aneurysm are considered. However, the description is also applicable to a thoracic aortic aneurysm. The following examples are illustrative only and are not intended to limit the RF heat treatment to stabilization of the aorta.

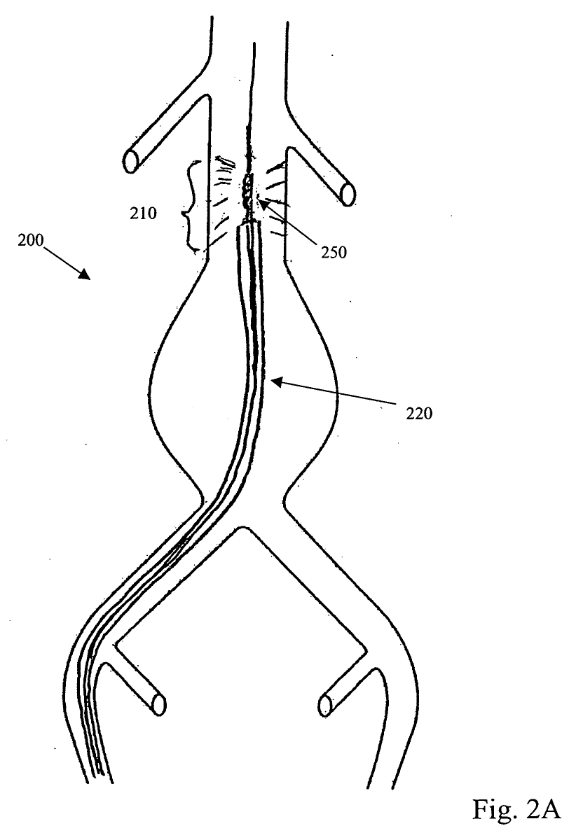

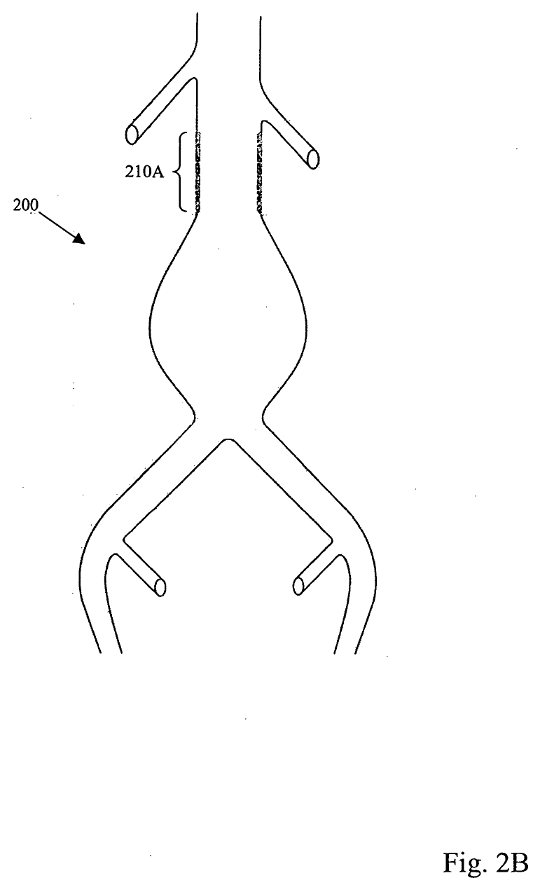

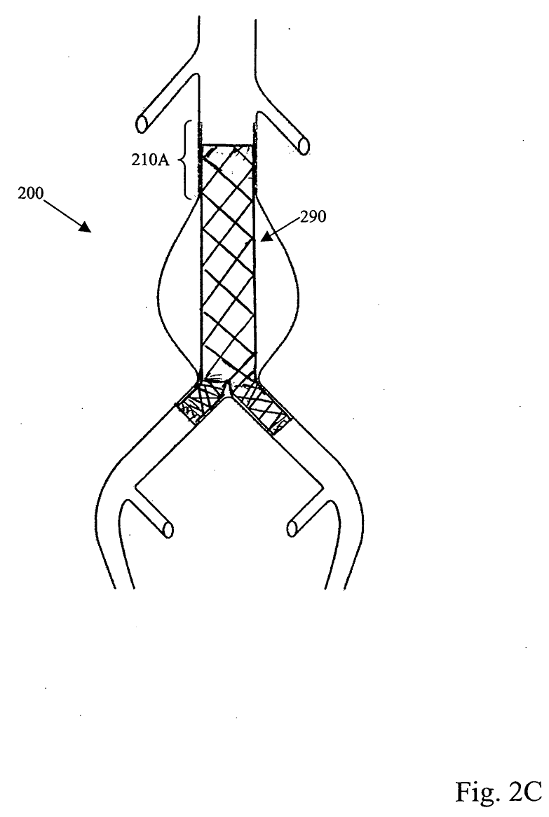

FIG. 2A is an illustration of an abdominal aortic aneurysm 200 prior to placement of a stent graft. A catheter 220 in inserted into aortic aneurysm 200 in a normal manner. A RF probe 250 is positioned in aortic neck 210 through catheter 220. Blood flow through the aorta is not blocked.

In this example, RF probe 250 is a bipolar RF probe. One example of RF probe 250 provides a focal beam, and another example provides a diffuse beam of RF energy. A particular shape and configuration of the antenna of RF probe 250 are selected to provide the desired radiation pattern, e.g., unidirectional or isotropic. The radiation pattern is selected that provides the best treatment of aortic neck 210.

For a unidirectional beam, RF probe 250 is placed adjacent a portion of aortic neck 210 and then maneuvered vertically and radially, as necessary, to treat aortic neck 210. For a particular RF probe 250 and radiation pattern, the RF energy and time of application necessary to obtain a specific shrinkage are, for example, determined empirically prior to use on patients.

The lines from RF probe 250 extend through catheter 220 out of the patient and are connected to a radio frequency generator/controller (not shown) that provides RF energy to RF probe 250. The RF generator/controller provides at least one of pulsed RF energy, square wave RF energy, sinusoidal wave RF energy and/or modulated RF energy. In one example, the frequency is in a range of 200 to 500 MHz, but other radio frequencies may also be used. The frequency is selected to provide a good depth of heating in aortic neck 210.

An example of a machine used to provide RF energy is the VAPR System of MITEK Products, a Division of ETHICON, Inc. (VARP and MITEK are trademarks of ETHICON, Inc., a Johnson and Johnson Company.) The VARP system includes a RF generator, a hand piece and cable, an electrode, and a footswitch.

After RF probe 250 is positioned in aortic neck 210, 20 watts are applied for 10 to 15 seconds, in one example. The time and power are selected so that aortic neck 210 is heated to a temperature above 60° C. for a time sufficient to shrink the collagen in aortic neck 210 and thereby stabilize aortic neck 210.

RF probe 250 can include a temperature sensor. In one example of the current method, up to 20 watts of power are delivered to the tissue being treated in aortic neck 210 until a temperature, near the temperature sensor, reaches between about 60° and 95° C. When the collagen reaches a temperature above its glass transition point, the collagen denatures and changes shape from a long linear protein to a globular protein. This change causes the collagen to shrink. Once some of the collagen has shrunk, more collagen is exposed to the RF energy. This collagen is heated and shrinks. Eventually, a steady state is reached where no further collagen shrinks based on the location of the heating element. This usually occurs within tens of seconds. If necessary, RF probe 250 is repositioned in aortic neck 210 and another region is treated

The blood flows by RF probe 250 and is not heated enough that coagulation becomes a problem. When aortic neck 210 is treated to obtain stabilized aortic neck 210A (FIG. 2B), RF probe 250 is removed. After stabilization, aortic neck 210A (FIG. 2A) has a reduced diameter compared to aortic neck 210 (FIG. 2A). Stent-graft 290 (FIG. 2C) is placed in aneurysm 200, now having stabilized aortic neck 210A, using the normal delivery procedure. As explained above, since aortic neck 210A is stabilized, the over sizing of stent graft 290 may be reduced or perhaps even eliminated depending on the amount of stabilization achieved.

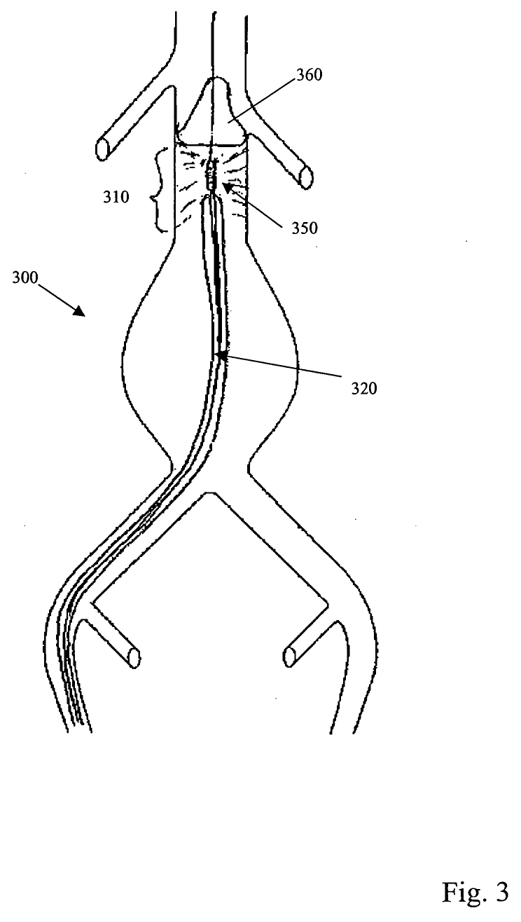

In the example of FIG. 2A, RF probe 250 functions in a liquid environment. In FIG. 3, RF probe 350 is positioned using catheter 320 in aortic neck 310. RF probe 350 is similar to RF probe 250 except RF probe 350 is not designed to function surrounded by a liquid environment. Thus, in this example, an occlusion balloon catheter 360 is also inserted to block blood flow through aortic neck 310. The other features and the operation of RF probe 350 are similar to those for probe 250 and so are not repeated.

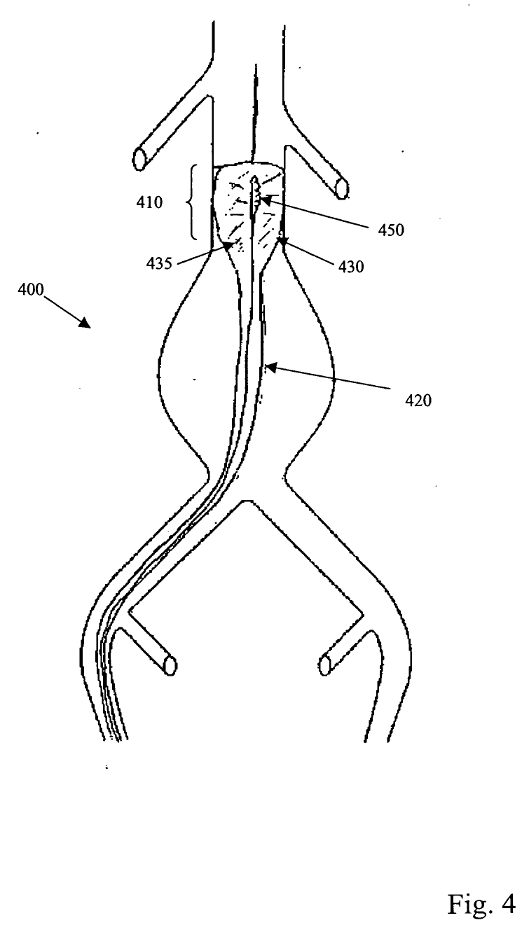

In the example of FIG. 4, RF probe 450 requires a particular liquid environment, e.g., a conductive environment such as that provided by saline, to provide the most efficient heating of aortic neck 410. Thus, at least a dual lumen catheter 420 is used. A balloon 430 is inserted in aortic neck 410. Balloon 430 is filled with required liquid 435 using one lumen to inflate the balloon. RF probe 450 is positioned inside inflated balloon 430 using the other lumen. The other features and the operation of RF probe 450 to stabilize aortic neck 410 of aneurysm 400 are similar to those for probe 250 and so are not repeated.

Balloon 430 is made of a compliant material so that balloon 430 contacts aortic neck 410. Also, in one example, the material of balloon 430 is selected to minimize the absorption so the RF energy so that most of the RF energy is deposited in aortic neck 410. Finally, the structural properties of the material are selected such that the RF environment does not adversely affect the functionality of balloon 430.

In another example, a proximal occlusion balloon and a distal occlusion balloon are used to isolate aortic neck 410. The use of dual occlusion balloons to isolate a region of a body lumen is known to those of skill in the art. After the two occlusion balloons are inflated and are in position, the necessary liquid is used to fill the volume between the two balloons. RF probe 450 is then used to heat aortic neck 410 to the desired temperature for the time required to obtain stabilization of aortic neck 410.

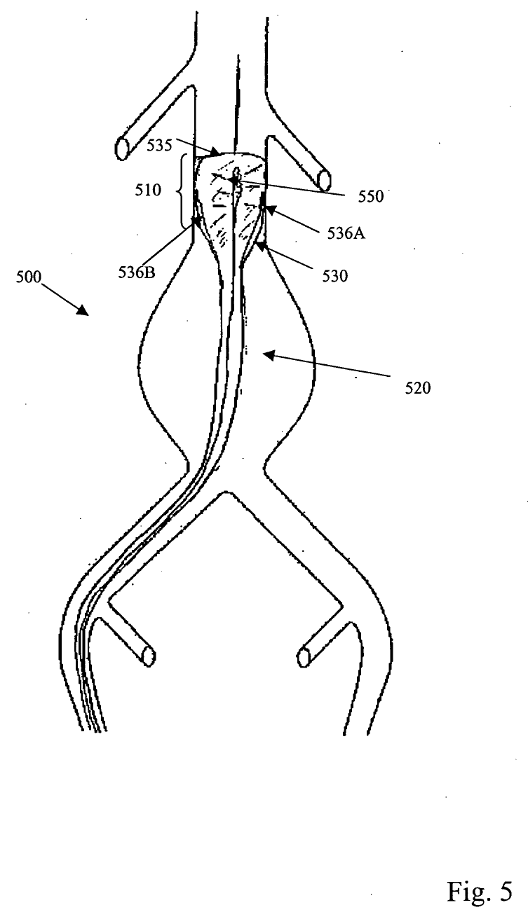

In the example of FIG. 5, RF probe 550 is similar to RF probe 450. RF probe 550 requires a particular liquid environment, e.g., a conductive environment such as that provided by saline, to provide the most efficient heating of aortic neck 510. Thus, a dual lumen catheter 520 is used. A balloon 530 is inserted in aortic neck 510. Balloon 530 is filled with required liquid 535 using one lumen. Balloon 530 is similar to balloon 430, except balloon 530 includes a plurality of attached temperature sensors 536A, 536B. RF probe 450 is positioned inside balloon 530 using the other lumen. The other features and the operation of RF probe 550 are similar to those for probe 250 and so are not repeated.

In one example, temperature sensors 536A and 536B are mounted on balloon 530 so that temperature sensors 536A and 536B are in direct contact with aortic neck 510 of abdominal aortic aneurysm. Temperature sensors 536A and 536B operate properly in an RF environment, and are thermally isolated from RF probe 550. Temperature sensors 536A and 536B operate properly irrespective of the orientation of RF probe 550 with respect to aortic neck 510. Finally, temperature sensors 536A and 536B have a temporal resolution sufficient to monitor rapid changes in temperature associated with the RF energy, such as when the RF energy is modulated. Connecting wires from the temperature sensors extend through catheter 520 to a measuring circuit, which in turn can be connected in a feedback loop to the RF generator. The feedback loop can be used to maintain the tissue temperature within a desired range.

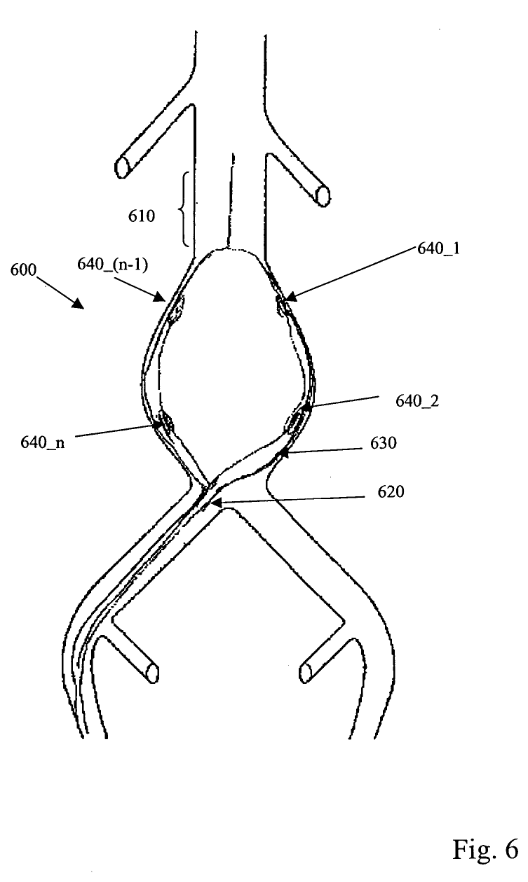

In the examples of FIGS. 2A to 2C and 3 to 5, the diameter of aortic neck is reduced by heating collagen in the aortic neck sufficiently to result in the shrinking and stabilization of the aortic neck. Similar advantages may be obtained by heating abdominal aortic aneurysm 600 itself using RF energy. In the example of FIG. 6, a plurality of RF elements 640_1 . . . 640_{—}n are arranged on a balloon 630 that is positioned in aneurysm 600 via catheter 620. Balloon 630 is inflated using a conventional technique so that plurality of RF elements 640_1 . . . 640_{—}n have contact with abdominal aortic aneurysm 600. RF power is supplied to plurality of RF elements 640_1 . . . 640_{—}n, as described above for probe 250, to heat the collagen in abdominal aortic aneurysm 600 to a temperature and for a time sufficient to cause aneurysm 600 to shrink. The shrinkage stabilizes aneurysm 600.

Plurality of RF elements 640_1 . . . 640_{—}n are arranged, in this example, in pairs where each pair functions as a bipolar RF element. The pairs are orientated about balloon to obtain a pattern of RF energy that heats aneurysm 600 to a temperature for a sufficient period to cause aneurysm 600 to shrink as the collagen shrinks. In another example, each element is a bipolar RF element and again the elements are orientated about balloon to obtain a pattern of RF energy that heats aneurysm 600 to a temperature for a sufficient period to cause aneurysm 600 to shrink as the collagen shrinks

Plurality of RF elements 640_1 . . . 640_{—}n are fabricated and then attached to balloon 630 in a manner similar to attaching an opaque marker to a balloon, e.g., bonded to a surface of the balloon. The pattern of the elements in plurality of RF elements 640_1 . . . 640_{—}n and the location of the elements on balloon 630 are selected not only to obtain the desired RF field pattern for heating, but also to permit collapsing balloon 630 for delivery via catheter 620.

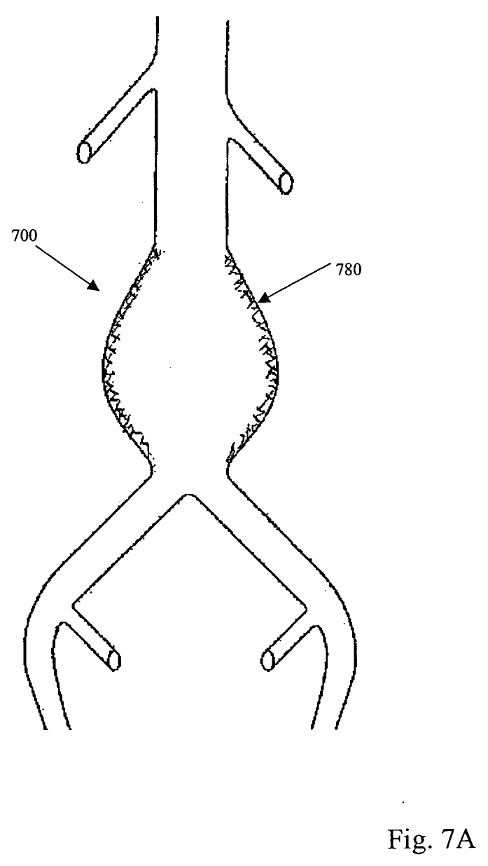

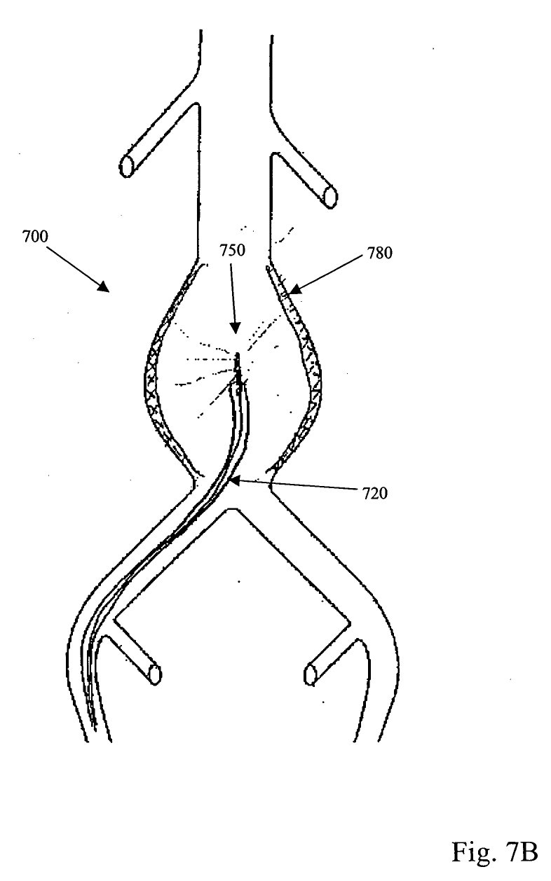

Another technique for shrinking and stabilizing an abdominal aortic aneurysm 700 (FIG. 7A ) is to insert a metallic mesh 780 adjacent to the inner wall of abdominal aortic aneurysm 700. One process for inserting metallic mesh 780 is described more completely below.

With metallic mesh 780 in place, a RF probe 750 (FIG. 7B) is inserted in abdominal aortic aneurysm 700 using catheter 720. Metallic mesh 780 and abdominal aortic aneurysm 700 are heated using RF probe 750. Metallic mesh 780 assists in distributing the heat more uniformly to abdominal aortic aneurysm 700.

As abdominal aortic aneurysm 700 shrinks in response to the collagen shrinking, metallic mesh 780 assists in strengthening the aortic wall. After treatment, a stent graft is installed in a normal manner. The metallic mesh is one of a braided mesh, a plain weave mesh, a twill-square weave mesh, a Hollander weave mesh or any mesh commonly used for a stent. The mesh is constructed to permit conformance of the mesh to the shape of aneurysm initially and as aneurysm 700 shrinks. The mesh is constructed of surgical grade metal such as nitinol or stainless steel, for example.

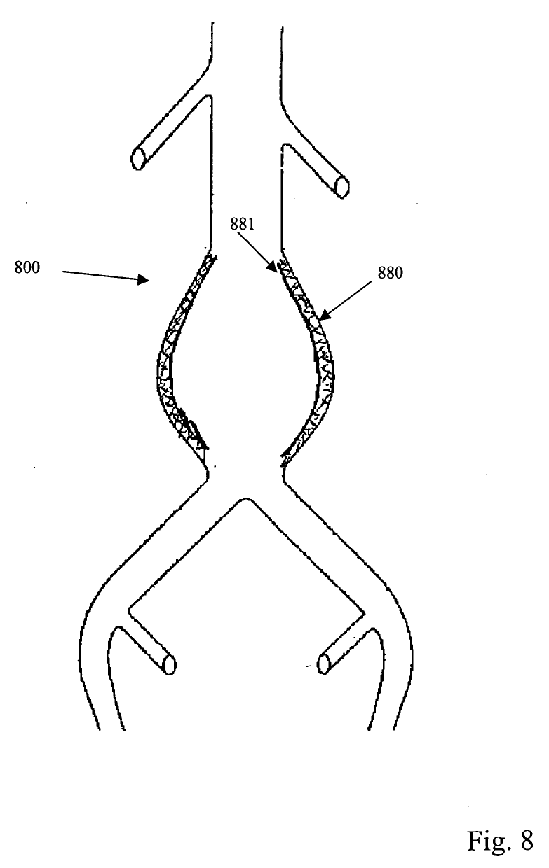

In another example, a metallic mesh 880 (FIG. 8) is attached to a layer of fabric 881 for support. Fabric 881 also assists in fitting mesh 880 in aortic aneurysm 800. Fabric 881 is, for example, a woven polyester material. Fabric 881 also could be a bioabsorbable material, a biodegradable material, polytetraflouroethylene (PTFE), polypropylene, polyethylene, polyurethane, and other materials known in the synthetic medical fabric device industry. The fabric selected is capable of acting as a temporary balloon during insertion of mesh 880. Also, if mesh 880 is used with a RF probe, the fabric is selected to withstand both the RF energy and the heat generated by absorption of the RF energy.

Another use of metallic mesh 780 and metallic mesh 880 is part of an in patient system for maintaining or recreating full embolization of the aneurysmal sac. The method utilizes electro coagulation to treat endoleaks.

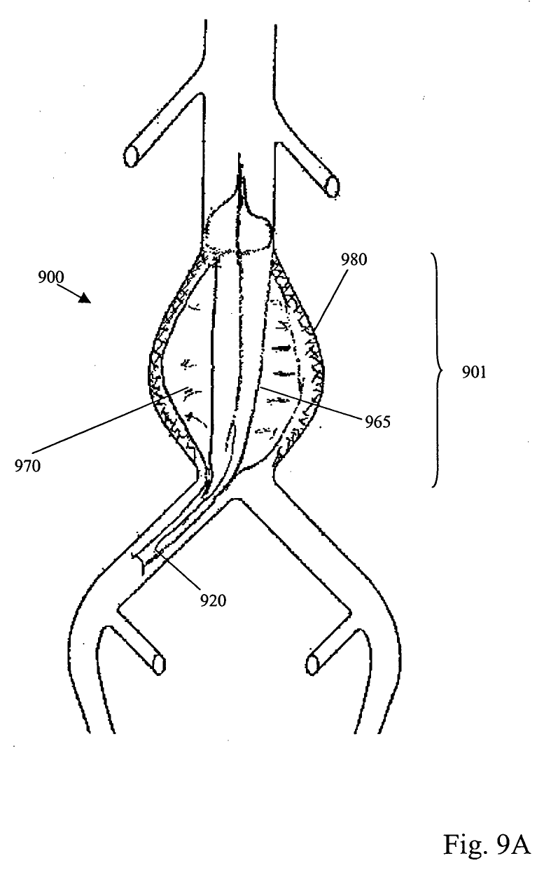

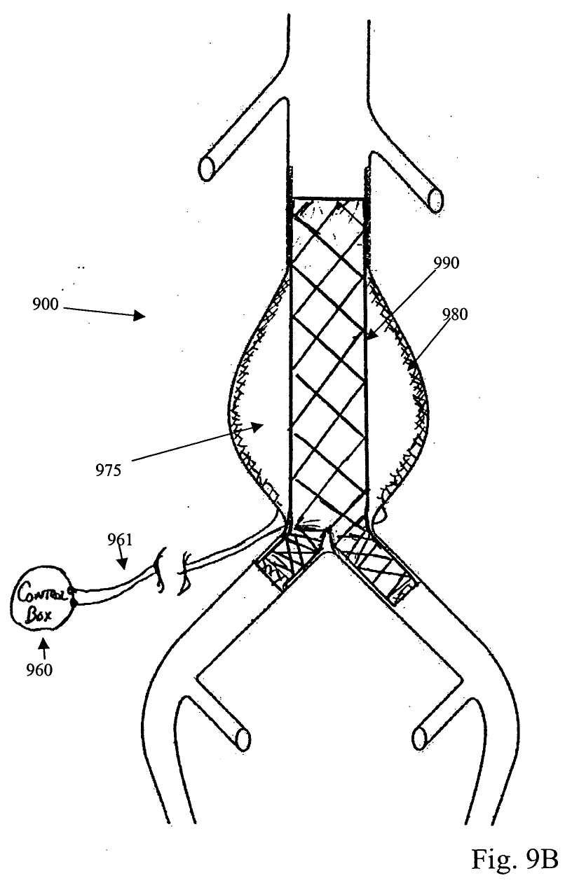

In method 1000, catheter 920 (FIG. 9A) is inserted and used to insert, for example, proximal and distal occlusion balloons that isolate aneurysm 900. After aneurysm 900 is isolated, aneurysmal sac 901 is cleaned in clean aneurysmal sac operation 1001. For example, a rinse agent is used to clean aneurysmal sac 9010.

After aneurysmal sac 901 is cleaned, metallic mesh 980 and balloon 965 are delivered to aneurysmal sac 901 via catheter 920 in deliver mesh operation 1002. Balloon 965 is used to maintain a channel for placement of the endograft.

Injecting a filling agent 970 in fill aneurysmal sac operation 1003 fills aneurysmal sac 901. The filing agent can be a gel, foam, pellets, or any other material suitable for use. Filling agent 970 expands metallic mesh 980 into contact with the wall of aneurysmal sac 901. Filing agent 970 fills all spaces.

Next, endograft 990 (FIG. 9B) is delivered in deliver endograft operation 1004. Alternatively, endograft 990 can be delivered first, and filling agent 970 can be injected second.

After endograft 990 is in place, the patient is opened to insert a control box 960 (FIG. 9B) in implant control box 1005. Control box 960 includes control circuitry and a power source for supply electrical current through metallic mesh 980 so that metallic mesh 980 is a resistive heater. Implantation of devices in the body is known. For example, pace makers and pumps used to deliver medication are implanted routinely in the body. The implantation of control box 960 follows an equivalent procedure.

Following and/or during the implantation of control box 960, leads 961 from metallic mesh 980 are connected to control box 960 in connect leads operation 1006. Again, the routing of the leads from metallic mesh 980 is similar to the procedure used in pacemaker implantation. After leads 961 are connected in connect leads operation 1006, the wound created to implant control box 960 is closed in close implantation operation 1007.

If a type II endoleak is detected using, for example, echo Doppler or radiography measurements, control box 960 is programmed to provide an electrical current to metallic mesh 980. The type II endoleak is immediately coagulated on site.

Various alternatives are possible. For example, metallic mesh 980 can be an array with parts of the array arranged so that conductivity or impedance of each part of the array can be measured. For example, leads from each part of the array are attached to an impedance change detection circuit so that in steady state with no endoleaks, the impedance of each part of the array is balanced with each of the other parts of the array. Thus, with no leaks, the impedances of the parts of the array are in balance. If a type II endoleak occurs, the impedances of the parts of the array are no longer balanced, and so control box applies an electrical current to either the entire array, or alternatively the part or parts of the array that experienced the impedance change to electro-coagulate the type II endoleak.

In another example, pressure sensors are attached to metallic array 980. A type II endoleak causes a pressure change that a pressure sensor, or pressure sensors detect. In response to the detected pressure change, control box 960 applies an electrical current to metallic mesh 980 sufficient to electro-coagulate the type II endoleak.

While in this example, metallic mesh 980 was inserted in aneurysmal sac 901, a similar procedure could be used to install a metallic mesh in the aortic neck. The metallic mesh in the aortic neck is used to electro-coagulate Type I endoleaks.

The above examples were for an aortic aneurysm in a human body. However, in view of this disclosure, the RF heat treatment process can be used for any vessel in a human or animal body where a stent graft or stent is used. Therefore, the above examples are illustrative only and are not intended to limit the invention to the specific examples used.

I claim:

1. A method comprising:
positioning a radio frequency probe in a body lumen; and
heating said body lumen, using said radio frequency probe, to stabilize said body lumen for insertion of a stent.

2. The method of claim 1 wherein said using said radio frequency comprises:
heating said body lumen to a temperature greater than 60 C.

3. The method of claim 1 further comprising:
allowing blood flow through said body lumen during said heating.

4. The method of claim 1 further comprising:
blocking blood flow through said body lumen during said heating.

5. The method of claim 1 wherein said body lumen comprises an aortic neck of an aortic aneurysm.

6. The method of claim 5 wherein said stent comprises a stent graft.

7. The method of claim 1 wherein said body lumen is an aortic aneurysm.

8. The method of claim 2 wherein said body lumen is an aortic aneurysm.

9. The method of claim 1 wherein said body lumen comprises an aneurysmal sac.

10. The method of claim 9 further comprising:
deploying a metallic mesh in said aneurysmal sac prior to said heating.

11. A method comprising:
positioning a radio frequency probe in a neck of an aortic aneurysm; and
heating said neck using said radio frequency probe to shrink a diameter of said neck.

12. The method of claim 11 further comprising:
inserting a stent-graft in said aortic aneurysm following said heating.

13. The method of claim 11 further comprising:
allowing blood flow through said neck during said heating.

14. The method of claim 11 further comprising:
blocking blood flow through said neck during said heating.

15. A method comprising;
inserting a metallic mesh in an aneurysmal sac of an aortic aneurysm; and
expanding said metallic mesh to contact a wall of said aneurysmal sac.

16. The method of claim 15 further comprising;
using said metallic mesh to electro-coagulate blood from a Type II endoleak.

17. The method of claim 15 further comprising:
delivering a stent graft to said aortic aneurysm.

18. The method of claim 15 wherein said expanding further comprises:
injecting a filler material into said aneurysmal sac.
